(19) United States
(12) Patent Application Publication
(10) Pub. No.: US 2026/0144641 A1
(43) Pub. Date: May 28, 2026

(54) Knee Prosthesis

(71) Applicant: The Third Hospital of Changsha, Changsha City (CN)

(72) Inventors: Weili ZHOU, Changsha City (CN); Qing LEI, Changsha City (CN); Tangyou LIU, Changsha City (CN); Xian YU, Changsha City (CN); Rong YU, Changsha City (CN)

(21) Appl. No.: 18/956,295

(22) Filed: Nov. 22, 2024

(51) Int. Cl. A61F 2/30; A61F 2/38

(52) U.S. Cl. CPC A61F 2/30771; A61F 2/3859; A61F 2/389; A61F 2002/30784; A61F 2002/30827

(57) ABSTRACT

Provided is a knee prosthesis. The knee prosthesis includes at least one of a femoral prosthesis, and a tibial prosthesis. The femoral prosthesis includes a first substrate, and a first connecting part connected to the first substrate. The tibial prosthesis includes a second substrate, and a second connecting part connected to the second substrate. A hard coating is arranged on at least one of outer surfaces of the first substrate and the second substrate.

### TECHNICAL FIELD

The present disclosure relates to the technical field of medical devices, and in particular to a knee prosthesis.

### BACKGROUND

With the improvement of surgical technology, the popularization of 3D printing technology and the use of robot navigation-assisted positioning, accurate and minimally invasive joint replacement with less bleeding and less bone removal volume is more and more used.

In prosthetic replacement, the unicompartmental knee replacement is one of the minimally invasive surgery for knee replacement at present. However, patients undergoing unicompartmental knee replacement only have anterior medial cartilage wear, which is characterized by pain when the knee joint is in an upright position and difficulty in walking. When conservative treatment does not work and prosthesis replacement is necessary, the volume of bone resection is still large. Especially, it is more difficult to restore the unicondylar knee prosthesis at the end of its life. In addition, at present, in order to avoid wear, most knee prostheses are made of heavy materials with high hardness, which is prone to increase the load of patients after surgery and has low comfort.

### SUMMARY

A main objective of the present disclosure is to provide a knee prosthesis, thus solving the problem of low comfort of a knee prosthesis in the prior art.

According to one aspect of the present disclosure, a knee prosthesis is provided, including at least one of a femoral prosthesis and a tibial prosthesis;
the femoral prosthesis includes a first substrate and a first connecting part connected to the first substrate;
the tibial prosthesis includes a second substrate and a second connecting part connected to the second substrate; and
a hard coating is arranged on at least one of outer surfaces of the first substrate and the second substrate.

The first substrate and/or the second substrate include/includes a titanium alloy substrate, the hard coating includes a titanium nitride coating.

Further, pores are formed in the first connecting part and/or the second connecting part.

Further, a volume of the pores on the first connecting part is 70% to 80% of a volume of the first connecting part; or
a volume of the pores on the second connecting part is 70% to 80% of a volume of the second connecting part.

Further, the pores include at least one of circular pores and square pores, and a diameter of each of the circular pores, or a side length of each of the square pores, ranges from 500 μm to 700 μm.

Further, each of the first connecting part and the second connecting part includes a connecting column, a first end of the connecting column is connected to the first substrate or the second substrate, a connecting hole is formed at a second end of the connecting column, the connecting hole extends in an axial direction of the connecting column, and a connector is detachably arranged inside the connecting hole.

Further, several grooves are formed on an end surface of the second end of the connecting column, the several grooves are arranged along a periphery of the connecting hole at intervals, and the several grooves extend in a radial direction of the connecting column.

Further, the several grooves each have a width of 0.5 mm to 1.5 mm.

Further, the connecting column includes a regular hexagonal prism, the regular hexagonal prism has a side length of 4.5 mm to 5.5 mm.

Further, the knee prosthesis further includes a plastic component; an annular groove is formed in the plastic component, and the plastic component is sleeved on the second substrate through the annular groove, and the plastic component and the second substrate are bonded and fixed by a nano-molding technology.

In the present disclosure, a hard coating is arranged on at least one of the first substrate and the second substrate. Through the arrangement of the hard coating, the wear efficiency when the first substrate and the second substrate are in contact and friction can be reduced. During actual design, a light material can be used to prepare the corresponding first substrate and second substrate, instead of using a heavy material with higher hardness to prepare the first substrate and the second substrate under the consideration of wear. Therefore, the load on the patient can be greatly reduced, and the service life and comfort of the knee prosthesis in the embodiments can be improved.

### BRIEF DESCRIPTION OF THE DRAWINGS

The accompanying drawings, which constitute a part of the present disclosure, are used to provide a further understanding of the present disclosure, and the illustrative embodiments of the present disclosure and their descriptions are used to explain the present disclosure, and do not constitute an undue limitation of the present disclosure. In the drawings:

FIG. 1 is a schematic diagram of a three-dimensional structure of a knee prosthesis according to an embodiment of the present disclosure;

FIG. 2 is a schematic diagram of a three-dimensional structure of a femoral prosthesis according to an embodiment of the present disclosure;

FIG. 3 is a bottom view of a femoral prosthesis according to an embodiment of the present disclosure;

FIG. 4 is a bottom view of a tibial prosthesis according to an embodiment of the present disclosure;

FIG. 5 is a schematic diagram of a three-dimensional structure of a tibial prosthesis according to an embodiment of the present disclosure;

FIG. 6 is a schematic diagram of a three-dimensional structure of a tibial prosthesis according to an embodiment of the present disclosure, with a plastic component removed;

FIG. 7 is a front view of a tibial prosthesis according to an embodiment of the present disclosure;

FIG. 8 is a front view of a plastic component according to an embodiment of the present disclosure; and

FIG. 9 is a structural schematic diagram when a connector according to an embodiment of the present disclosure is connected to a second end surface (pores not shown) of a connecting column.

The above described accompanying drawings include the following reference numerals:
10 femoral prosthesis; 11 first substrate; 12 first connecting part; 20 tibial prosthesis; 21 second substrate; 22 second connecting part; 30 pore; 121 connecting column; 123 connecting hole; 124 groove; 40 plastic component; 41 annular groove; 50 connector.

### DETAILED DESCRIPTION OF THE EMBODIMENTS

It should be noted that the embodiments in the present disclosure and the features in the embodiments can be combined with each other without conflict. The present disclosure is described in detail below with reference to the accompanying drawings and embodiments.

It should be noted that the terms used herein is only for describing specific embodiments, and is not intended to limit exemplary embodiments according to the present disclosure. As used herein, unless the context clearly indicates otherwise, the singular form is also intended to include the plural form. Furthermore, it should be understood that when the terms “comprising”, “including” are used in this specification, they indicate the presence of features, steps, operations, devices, assemblies, and/or their combinations.

Unless otherwise specified, the relative arrangement of parts and steps, numerical expressions and numerical values set forth in these embodiments are not intended to limit the scope of the present disclosure. Meanwhile, it should be understood that for the convenience of description, the dimensions of various parts shown in the accompanying drawings are not drawn according to the actual scale relationship. Technologies, methods and devices known to those of ordinary skill in the related art may not be discussed in detail, but such technologies, methods and devices should be considered as a part of the specification in appropriate situations. In all examples shown and discussed herein, any specific value should be interpreted as illustrative only, rather than limiting. Therefore, other examples of exemplary embodiments may have different values. It should be noted that similar symbols and letters indicate similar items in the following accompanying drawings, so once an item is defined in one accompanying drawing, it does not need to be further discussed in subsequent drawings.

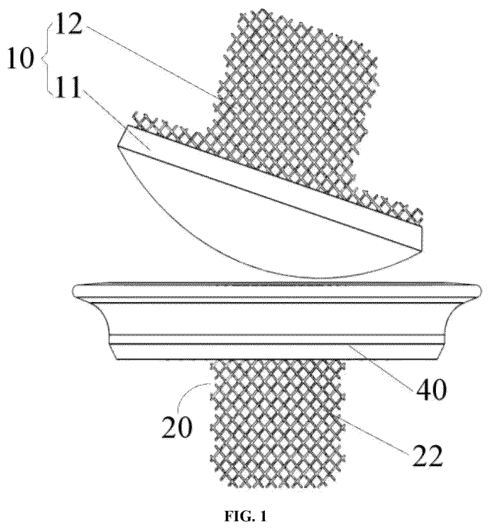

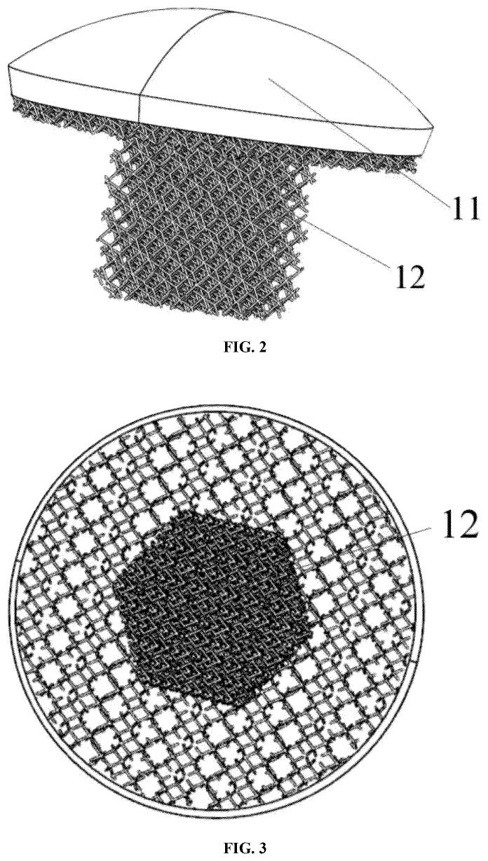

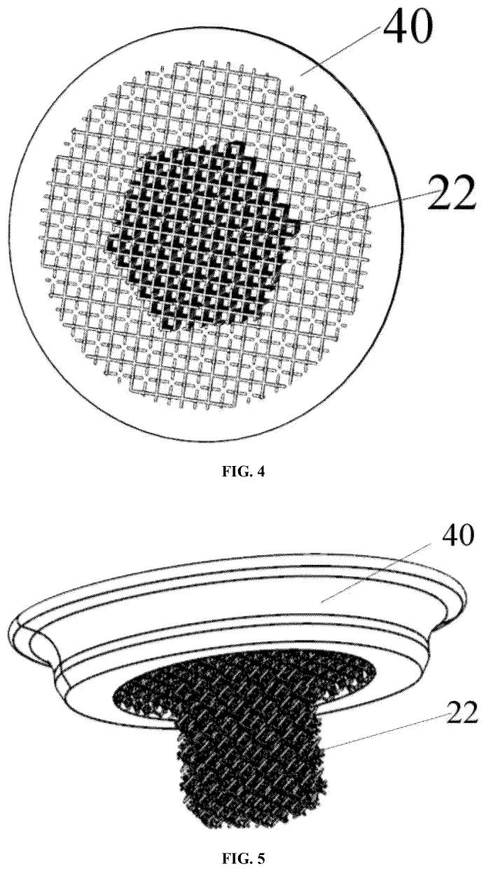

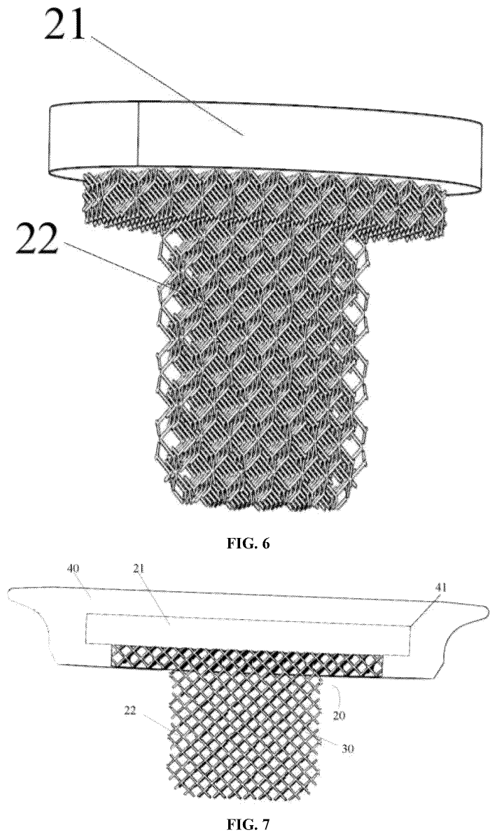

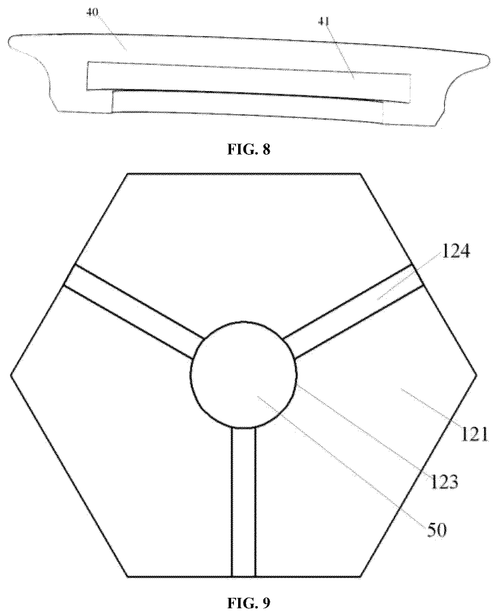

Referring to FIG. 1 to FIG. 9, a knee prosthesis is provided according to an embodiment of the present disclosure. The knee prosthesis includes at least one of a femoral prosthesis 10 and a tibial prosthesis 20.

The femoral prosthesis 10 includes a first substrate 11 and a first connecting part 12 connected to the first substrate 11. The tibial prosthesis 20 includes a second substrate 21 and a second connecting part 22 connected to the second substrate 21. A hard coating (not shown in the figure) is arranged on at least one of outer surfaces of the first substrate 11 and the second substrate 21.

During actual surgery, the first substrate 11 of the femoral prosthesis 10 can be connected to femur through a connection function of the first connecting part 12. The second substrate 21 of the tibial prosthesis 20 is connected to tibia through a connection function of the second connecting part 22.

In the present disclosure, at least one of the first substrate 11 and the second substrate 21 is provided with the hard coating. Through the arrangement of the hard coating, the wear efficiency when the first substrate 11 and the second substrate 21 are in contact and friction can be reduced. During actual design, a light material can be used to prepare the corresponding first substrate 11 and second substrate 21, instead of using a heavy material with higher hardness to prepare the first substrate 11 and the second substrate 21 under the consideration of wear. Therefore, the load on the patient can be greatly reduced, and the service life and comfort of the knee prosthesis in this embodiment can be improved.

It should be noted that the knee prosthesis in this embodiment is especially suitable for an articular cartilage wear site which is not larger than 20 mm. During actual surgery, only one, or both, of the femoral prosthesis 10 and the tibial prosthesis 20 in this embodiment can be implanted. That is, the femoral prosthesis 10 and the tibial prosthesis 20 can be used alone or in combination, depending on a lesion site and lesion condition of the patient.

Exemplary, the first substrate 11 and/or the second substrate 21 include/includes a titanium alloy substrate, and the hard coating includes a titanium nitride coating. That is, in this embodiment, the first substrate 11 can be a titanium alloy substrate, or the second substrate 21 can be a titanium alloy substrate, or both the first substrate 11 and the second substrate 21 can be titanium alloy substrates. The titanium alloy has light weight, and good heat resistance, strength, plasticity, formability, corrosion resistance and bio-compatibility. The knee prosthesis, when used in the knee joint, is more suitable to grow and fuse with the human knee joint. The titanium alloy has extremely high hardness, and good heat resistance, corrosion resistance and wear resistance. In this embodiment, by arranging the hard coating as a titanium nitride coating, metal ions on the femoral prosthesis 10 and the tibial prosthesis 20 can be effectively prevented from precipitating, and meanwhile, the trouble that titanium alloy cannot be used as a friction interface is avoided.

It should be noted that in this embodiment, the first substrate 11 and the second substrate 21 have a relatively light weight structure. Besides the titanium alloy, the first substrate 11 and the second substrate 21 may be cobalt alloy, tantalum, niobium, platinum, etc.

During actual processing and design, the surfaces of the femoral prosthesis 10 and the tibial prosthesis 20 are constructed by using CT (computed tomography) and a three-dimensional reconstruction technology to conform to a curvature structure of the knee joint surface, such that the first substrate 11 and the second substrate 21 can adapt to the movement of the knee joint. Alternatively, in this embodiment, the first substrate 11 is spherical, or ellipsoidal, and can be made by three-dimensional reconstruction and 3D printing technology according to the lesion location of the patient. The second substrate 21 is disc-shaped, other variations of which under the concept of the present disclosure are within the scope of protection of the present disclosure.

Further, pores 30 are formed in the first connecting part 12 and/or the second connecting part 22. That is, in this embodiment, the pores 30 may be formed in the first connecting part 12, or the pores 30 may be formed in the second connecting part 22, or the pores 30 may be formed in both the first connecting part 12 and the second connecting part 22. In the present disclosure, a case that the pores 30 are formed in both the first connecting part 12 and the second connecting part 22 is preferably adopted. The arrangement of the pores 30 is more suitable for osteocytes to grow in and fuse with the knee prosthesis.

Alternatively, in this embodiment, the occupancy of the pores 30 is 70% to 80%, such as 70%, 72%, 74%, 75%, 76%, 78% and 80%. That is, the sum of the volume of the pores 30 on the first connecting part 12 is 70%-80% of the volume of the first connecting part 12, and the sum of the volume of the pores 30 on the second connecting part 22 is 70%-80% of the volume of the second connecting part 22. When the occupancy of the pores 30 is less than 70%, osteocytes are not conducive to grow and fuse with the knee prosthesis. When the occupancy of the pores 30 is greater than 80%, the structural strength and connection stability of the first connecting part 12 and the second connecting part 22 are prone to reduced.

Exemplary, the pores 30 includes at least one of circular pores and square pores, and a diameter of the circular pore, or a side length of the square pore, ranges from 500 μm to 700 μm, such as 500 μm, 550 μm, 600 μm, 650 μm, or 700 μm. When the diameter of the circular pore or the side length of the square pore is less than 500 μm, processing forming is inconvenient, the production and processing cost of the knee prosthesis is prone to increase. When the diameter of the circular pore or the side length of the square pore is greater than 700 μm, the pore 30 is relatively large, which is inconvenient for the growth and fusion of the osteocytes and knee prosthesis. That is, in this embodiment, by making the diameter of the circular pore or the side length of the square pore range from 500 μm to 700 μm, the processing cost of the knee prosthesis and the fusion speed of the osteocytes with the knee prosthesis can be taken into account.

Refer to FIG. 1 to FIG. 9, each of the first connecting part 12 and the second connecting part 22 includes a connecting column 121. A first end of the connecting column 121 is connected to the first substrate 11 or the second substrate 21. A connecting hole 123 is formed in a second end of the connecting column 121. The connecting hole 123 extends in an axial direction of the connecting column 121, and a connector 50 is detachably arranged inside the connecting hole 123. During surgery, the first connecting part 12 and the second connecting part 22 can be pre-positioned on the knee joint of the human body by the connector 50. Exemplary, the connector 50 in this embodiment may be a PE (polyethylene) bar, or a PI (polyimide) bar.

Alternatively, several grooves 124 are formed on an end surface of the second end of the connecting column 121, the several grooves 124 are arranged along the periphery of the connecting hole 123 at intervals, and the grooves 124 extend in a radial direction of the connecting column 121. During actual design, there may be one, two, three or more grooves 124 in this embodiment. During mounting, after the connector 50 is implanted in advance, the tibial prosthesis 20 or the femoral prosthesis 10 is implanted. When the connector 50 is inserted into the connecting hole 123, the connecting column 121 may expand at a position of the grooves, the connector 50 can effectively squeeze the connecting column 121 to generate a pre-stress between the connecting column 121 and the bone, such that the first connecting part 12 and the second connecting part 22 can be stably implanted into the knee joint of the human body.

In this embodiment, the groove 124 has a width ranging from 0.5 mm to 1.5 mm, such as 0.5 mm, 0.7 mm, 0.9 mm, 1.1 mm, 1.3 mm, or 1.5 mm. When the width of the groove 124 is less than 0.5 mm, and the first connecting part 12 or the second connecting part 22 is implanted into the connector 50, the deformability of the connecting column 121 is weak, which is inconvenient to apply pre-stress to the connecting column 121 to connect the first connecting part 12 and the second connecting part 22 to the knee joint of a human body. When the width of the groove is greater than 1.5 mm, and the first connecting part 12 or the second connecting part 22 is implanted into the connector 50, the connecting column 121 has large deformation, which is prone to affect the structural strength and connection stability of the connecting column 121.

Further, the connecting column 121 is a regular hexagonal prism which has a side length ranging from 4.5 mm to 5.5 mm, such as 4.5 mm, 5 mm, or 5.5 mm. Such an arrangement can make the knee prosthesis in this embodiment suitable for a patient with articular cartilage wear not more than 20 mm. Certainly, in other embodiments of the present disclosure, the connecting column 121 may also be a cylinder, a square column, an oval column, or other special-shaped columnar structure, and other variations of the connecting column under the concept of the present disclosure are within the scope of protection of the present disclosure.

Further, the knee prosthesis further includes a plastic component 40. An annular groove 41 is formed in the plastic component 40. The plastic component 40 is sleeved on the second substrate 21 through the annular groove 41. The plastic component 40 and the second substrate 21 are bonded and fixed by a nano-molding technology. The plastic component 40 is generally in the form of an annular sleeve structure, which is sleeved on the periphery of the second substrate 21 to provide a friction interface for the tibial prosthesis 20. Since the plastic component 40 in this embodiment is bonded to the second substrate 21 by the nano-molding technology, secondary clamping and positioning can be avoided, and the friction on interface of the plastic and the metal on the tibial prosthesis 20 can be removed. During actual processing, the surface of the plastic component 40 also needs to be constructed by using CT and three-dimensional reconstruction to conform to a body curvature structure of a distal femoral surface, such that the surface curvature of the plastic component 40 is suitable for the movement of the femur and tibia. The plastic component 40 is provided with an annular groove 41, and the annular groove 41 is in fit with the second substrate 21 to prevent the second substrate 21 from separating. Nano Molding Technology (NMT) is a process of combining metal and plastic by nanotechnology, that is, the surface of the metal is subjected to nano treatment, and then the plastic is directly injected and molded on the surface of the metal, such that the metal and the plastic can be integrally molded and finally combined into one product. The “nano” referred to here refers to a microporous process, that is, the surface of metal is subjected to nano-scale microporous treatment through a specific solution, and the main purpose is to better combine the surface of the metal with the plastic, thus improving the connection strength.

As can be known from the above description that at edge parts of the femoral prosthesis 10 and the tibial prosthesis 20, the human tissue configuration is reconstructed by CT and three-dimensional technology to generated a curved surface of the prosthesis. During actual surgery, in order to ensure the accuracy of prosthesis implantation, the prosthesis is precisely implanted by using a surgical robot. The knee joint substrate provided by the present disclosure can be suitable for an articular cartilage wear site which is not larger than 20 mm, which can greatly reduce the hidden danger load on the patient, and has long service life and good use comfort.

For the convenience of description, spatial relative terms such as “above”, “over”, “on the upper surface of” and “upper” can be used here to describe a spatial positional relationship between a device or feature and other devices or features as shown in the figure. It should be understood that the spatial relative terms are intended to encompass different orientations in use or operation in addition to the orientation of the device depicted in the accompanying drawings. For example, if the devices in the accompanying drawings are inverted, devices described as “over other devices or structures” or “above other devices or structures” will be positioned as “under other devices or structures” or “below other devices or structures”. Therefore, the exemplary term “over” may include both directions of “over” and “under”. The device may also be positioned in other different ways (rotated by 90 degrees or in other orientations), and the spatial relative description used herein is explained accordingly.

In addition, it should be noted that the words “first” and “second” are used to define parts only for the convenience of distinguishing the corresponding parts. Unless otherwise stated, the above words have no special meaning, and thus cannot be construed as limiting the scope of protection of the present disclosure.

The above is only the preferred embodiment of the present disclosure, and is not used to limit the present disclosure. For those skilled in the art, the present disclosure may have various modifications and changes. Any modification, equivalent substitution, improvement, etc. made within the spirit and principle of the present disclosure shall fall within the scope of protection of the present disclosure.

What is claimed is:

1. A knee prosthesis, comprising at least one of a femoral prosthesis (10) and a tibial prosthesis (20);
wherein the femoral prosthesis (10) comprises a first substrate (11) and a first connecting part (12) connected to the first substrate (11);
the tibial prosthesis (20) comprises a second substrate (21) and a second connecting part (22) connected to the second substrate (21); and
wherein a hard coating is arranged on at least one of outer surfaces of the first substrate (11) and the second substrate (21).

2. The knee prosthesis according to claim 1, wherein at least one of the first substrate (11) and the second substrate (21) comprises a titanium alloy substrate, and the hard coating comprises a titanium nitride coating.

3. The knee prosthesis according to claim 1, wherein pores (30) are formed in at least one of the first connecting part (12) and the second connecting part (22).

4. The knee prosthesis according to claim 3, wherein a volume of the pores (30) on the first connecting part (12) is 70% to 80% of a volume of the first connecting part (12); or
a volume of the pores (30) on the second connecting part (22) is 70% to 80% of a volume of the second connecting part (22).

5. The knee prosthesis according to claim 3, wherein the pores (30) comprise at least one of circular pores and square pores, and a diameter of each of the circular pores, or a side length of each of the square pores, ranges from 500 μm to 700 μm.

6. The knee prosthesis according to claim 1, wherein each of the first connecting part (12) and the second connecting part (22) comprises a connecting column (121), a first end of the connecting column (121) is connected to the first substrate (11) or the second substrate (21), a connecting hole (123) is formed at a second end of the connecting column (121), the connecting hole (123) extends in an axial direction of the connecting column (121), and a connector (50) is detachably arranged inside the connecting hole (123).

7. The knee prosthesis according to claim 6, wherein several grooves (124) are formed on an end surface of the second end of the connecting column (121), the several grooves (124) are arranged along a periphery of the connecting hole (123) at intervals, and the several grooves (124) extend in a radial direction of the connecting column (121).

8. The knee prosthesis according to claim 7, wherein the several grooves (124) each have a width of 0.5 mm to 1.5 mm.

9. The knee prosthesis according to claim 6, wherein the connecting column (121) comprises a regular hexagonal prism, the regular hexagonal prism has a side length of 4.5 mm to 5.5 mm.

10. The knee prosthesis according to claim 1, wherein the knee prosthesis further comprises a plastic component (40); an annular groove (41) is formed in the plastic component (40), and the plastic component (40) is sleeved on the second substrate (21) through the annular groove (41), and the plastic component (40) and the second substrate (21) are bonded and fixed by a nano-molding technology.

11. The knee prosthesis according to claim 2, wherein the knee prosthesis further comprises a plastic component (40); an annular groove (41) is formed in the plastic component (40), and the plastic component (40) is sleeved on the second substrate (21) through the annular groove (41), and the plastic component (40) and the second substrate (21) are bonded and fixed by a nano-molding technology.

12. The knee prosthesis according to claim 3, wherein the knee prosthesis further comprises a plastic component (40); an annular groove (41) is formed in the plastic component (40), and the plastic component (40) is sleeved on the second substrate (21) through the annular groove (41), and the plastic component (40) and the second substrate (21) are bonded and fixed by a nano-molding technology.

13. The knee prosthesis according to claim 4, wherein the knee prosthesis further comprises a plastic component (40); an annular groove (41) is formed in the plastic component (40), and the plastic component (40) is sleeved on the second substrate (21) through the annular groove (41), and the plastic component (40) and the second substrate (21) are bonded and fixed by a nano-molding technology.

14. The knee prosthesis according to claim 5, wherein the knee prosthesis further comprises a plastic component (40); an annular groove (41) is formed in the plastic component (40), and the plastic component (40) is sleeved on the second substrate (21) through the annular groove (41), and the plastic component (40) and the second substrate (21) are bonded and fixed by a nano-molding technology.

15. The knee prosthesis according to claim 6, wherein the knee prosthesis further comprises a plastic component (40); an annular groove (41) is formed in the plastic component (40) and the plastic component (40) is sleeved on the second substrate (21) through the annular groove (41), and the plastic component (40) and the second substrate (21) are bonded and fixed by a nano-molding technology.

16. The knee prosthesis according to claim 7, wherein the knee prosthesis further comprises a plastic component (40); an annular groove (41) is formed in the plastic component (40), and the plastic component (40) is sleeved on the second substrate (21) through the annular groove (41), and the plastic component (40) and the second substrate (21) are bonded and fixed by a nano-molding technology.

17. The knee prosthesis according to claim 8, wherein the knee prosthesis further comprises a plastic component (40); an annular groove (41) is formed in the plastic component (40), and the plastic component (40) is sleeved on the second substrate (21) through the annular groove (41), and the plastic component (40) and the second substrate (21) are bonded and fixed by a nano-molding technology.

18. The knee prosthesis according to claim 9, wherein the knee prosthesis further comprises a plastic component (40); an annular groove (41) is formed in the plastic component (40), and the plastic component (40) is sleeved on the second substrate (21) through the annular groove (41), and the plastic component (40) and the second substrate (21) are bonded and fixed by a nano-molding technology.
